(19) United States
(12) Patent Application Publication
(10) Pub. No.: US 2002/0107213 A1
(43) Pub. Date: Aug. 8, 2002

(54) Gene therapy of alzheimer's disease by delivery of an encoded apoliprotein E

(76) Inventors: Stefan Frederik F. Verlinden, Leiden (NL); Dirk Willem van Bekkum, Rotterdam (NL)

Correspondence Address: TRASK BRITT, P.O. BOX 2550, SALT LAKE CITY, UT, 84110, US

(21) Appl. No.: 09/835,647

(22) Filed: Apr. 16, 2001

Related U.S. Application Data
(63) Continuation of application No. PCT/NL99/00638, filed on Oct. 15, 1999.

(30) Foreign Application Priority Data
Oct. 16, 1998 (EP) 98203480.3

(51) Int. Cl. A61K 48/00

(52) U.S. Cl. 514/44

(57) ABSTRACT

Gene therapy vehicles to alter the ApoE phenotype of human cells. The ApoE phenotype of the glial cells in the brain is related to the risk of development of dementia such as Alzheimer's disease.

### CROSS REFERENCE TO RELATED APPLICATIONS

This application is a continuation of co-pending International Application PCT/NL99/00638, filed Oct. 15, 1999, designating the United States of America, which itself claims priority from EP 98203480.3, filed on Oct. 16, 1998, the contents of all these applications are incorporated by this reference in their entirety.

### TECHNICAL FIELD

The invention relates to Alzheimer's disease and more specifically to gene therapy and prevention of Alzheimer's disease.

### BACKGROUND

Alzheimer's disease (AD) is the leading cause of dementia in the elderly, affecting approximately 5% of the population over age 65, which figure rises to >40% for the population over 85 years old. Symptoms of AD and dementia in general are progressive cognitive decline, in particular loss of memory, learning, and attention. Upon pathological examination, accumulation of amyloid β (Aβ) in the brain is found. Other forms of dementia that have the development of amyloid plaques in common are cerebral amyloid angiopathy (CAA) and vascular dementia or AD with cerebrovascular disease.

The neuropathology of AD is characterized by extensive neuronal cell loss and deposition of numerous senile plaques and neurofibrillary tangles in the cerebral cortex. The major component of the senile plaques is Aβ, a 39 to 43 amino acid peptide derived by proteolytic cleavage from the amyloid β precursor protein (APP). Soluble Aβ species of different lengths are physiologically present in various body fluids, including cerebrospinal fluid. The Aβ deposits found in the brain occur as extracellular diffuse plaques, as well as neuritic plaques containing dense cores surrounded by dystrophic neurites. Neurofibrillary tangles are found intraneuronally and are composed mainly of paired helical filaments containing a hyperphosphorylated form of the microtubule-associated protein tau. Regions that are affected most are the temporal lobes and the frontal, parietal, and posterior cingulate cortices, which areas are associated with cognitive functions.

The underlying disease mechanism is not yet understood although some genetic linkage is known, which led to the finding of two distinct forms, early and late onset AD.

1) Early onset or familiar AD. Five percent of all AD patients develop disease before age 60. In these patients, mutations of the APP, the presenilin1 gene or the presenilin2 gene located on chromosomes 21, 14 or 1 are found. These mutations have in common that they alter APP or Aβ processing, which results in plaque formation and AD.

2) Late onset or sporadic AD, which affects the vast majority of all patients, with a disease onset after age 60. In these patients a linkage to a region on chromosome 19 was shown, where the apolipoprotein E (APOE for the gene and ApoE for the protein) locus is found (Pericak-Vance et al. 1991).

ApoE is a 34 kDa plasma protein, that binds to the low-density lipoprotein receptor and/or the LDL receptor-related protein a/2-macroglobulin receptor (LRP) and is involved in the transport of cholesterol and other lipids in various cells in the body. The LRP receptor is present on brain neurons. Mutational analysis showed that region 136-160 of ApoE is critical for LDL receptor interaction.

The majority of the plasma ApoE is derived from the liver, where it is synthesized by hepatocytes. In other organs and tissues, it is also synthesized, albeit in much lower levels, by monocyte-derived macrophages. In different organs/tissues, macrophages have different names, for instance, microglial cells in the brain. In the brain, after the liver the organ that produces the second most ApoE, the protein is synthesized by glial cells, of which originally only microglial cells were thought to be monocyte-derived. Recently it was shown that macroglial cells are also monocyte-derived (Eglitis and Mezey, 1997), so all glial cells are monocyte-derived.

ApoE is found in humans by isoelectric focusing in three major isoforms (E2, E3 and E4), minor isoforms in humans are E1, E5, E6 and E7. Within these isoforms different mutations can be found that result in yet further differing proteins having the same isoelectric focusing point (Weisgraber 1994). Some examples of isoform mutations are ApoE2-Christchurch, ApoE2-Heidelberg, ApoE1-Harrisburg, ApoE4-Philadelphia, and ApoE3-Leiden (de Villiers, 1997).

The three major allelic variants are found in frequencies of 77% for e3, 15% for e4, and 7% for e2, differing by single amino acid substitutions at positions 112 and 158: e3 (Cys 112, Arg 158), e4 (Cys 112Arg), and e2 (Arg158Cys).

Further studies in late onset AD patients revealed that the frequency of APOE e4 is increased in AD patients and that there is a gene dose effect (Strittmater et al., 1993). Not only is the risk for AD increased significantly, but the mean age of onset of AD also decreases with an increasing number of APOE e4 alleles from >75 for 0 alleles to <70 when two alleles are present (Higgins, 1997). It also was found that subjects carrying one or two APOE e2 alleles are protected from the disease, and if they get AD, the mean age of onset is >80 years of age. When the chance of getting AD in the age group 60-65 is set to 1 for subjects with the phenotype e3/e3, the chance is 0.1 for subjects with the phenotype e2/e3, 1.1 with e2/e4, 11.1 with e3/e4 and 123.8 with e4/e4 (Corder, 1994).

It is suggested that ApoE, amongst others, is involved in the clearing of Aβ (Aleshkov, 1997). The idea is that AD is actually a processing disorder. This idea seems to be supported by the finding that AD occurs in Down syndrome patients without mutations in APP, presinilin1, or presenilin2, with a disease onset before age 60. Due to trisomy of chromosome 21, which contains the APP gene, Down syndrome patients express 1.5 times the amount of APP as compared to normal humans. Typically, these patients start developing AD from age 40. In different studies, both an increased risk for AD when one or two APOE e4 alleles are present (Schupf, 1998) and a protective effect when one or two APOE e2 alleles are found (Tyrrel, 1998). In conclusion, enhancement of APP, the Aβ precursor protein, leads to earlier onset of AD with a similar distribution of APOE phenotypes in Down syndrome patients and normal persons.

Human primates are the only species known to possess at least three different APOE isotypes. All other known species, except rabbits, which carry APOE e3, only carry the APOE e4 isotype (Poduri, 1992). From the group of non-human primates, only Rhesus monkeys were described to develop AD-like pathology at age >25 years. This is a major obstruction in the study of late onset AD, because no easily accessible animal model is available and a study in Rhesus monkeys would require several decades, depending on the strategy. The study of early AD is possible in rodents and other small animals, since the genes with the same mutations found in men (i.e. APP, presenilin1, or presenilin2) when introduced in transgenic mice results in similar pathology. However, the observed APOE gene in man is not found in mice, so it seems that the mode of action of ApoE in mice differs from that found in man. Recently it was found that in APOE −/− knockout mice, age related congophilic inclusions in the brain occur that are to a much lesser extent present in APOE+/+ parental mice (Robertson, 1998). These congophilic inclusions consist of ubiquitin and Aβ and are found predominantly in protoplastic astrocytes or macroglial cells in the dorsal hippocampus, a location were one expects to find plaques in AD patients. Other areas where congophilic inclusions are found are the piriform cortex and cerebellum.

### SUMMARY OF THE INVENTION

The present invention relates to the field of human gene therapy, more particularly to novel gene therapy vehicles to alter the ApoE phenotype of human cells. As described herein, the ApoE phenotype of the glial cells in the brain is related to the risk of development of dementia such as Alzheimer's disease. For the purpose of allowing gene therapy aimed at the reduction of this risk, the invention provides gene therapy vehicles suitable to alter the ApoE phenotype of human cells.

The invention provides a gene delivery or gene therapy vehicle and methods for its use in the treatment and prevention of dementia, such as Alzheimer's disease, wherein a functional APOE gene or a functional equivalent thereof is introduced or delivered into human or humanized cells, such as progenitor cells, for example, monocytes or glial cells. Monocytes and subsequently glial cells originate from hematopoietic stem cells in the bone marrow. It has been shown that bone marrow transplantations may lead to complete and long-lasting chimerism of hematopoietic stem cells and their progeny. In the host, monocyte-derived tissue macrophages (e.g., glial cells) will be replaced by donor macrophages. This technique is used, for instance, to replace glial cells in patients with lysosomal storage diseases based on an enzyme defect by bone marrow transplantation with macrophages from a healthy donor that contain the correct genetic information for the affected enzyme (Hoogerbrugge, 1988). Similar experiments have been performed in atherosclerotic mice. Since the main systemic function of ApoE is clearance of lipoproteins from the plasma, APOE −/− mice also develop hyperlipoproteinemia. High lipoprotein levels cause precipitation and deposition of these proteins on the artery wall, leading to atheroscleroses probably due to inadequate processing by local tissue macrophages. Transplanting bone marrow from APOE +/+ mice into APOE −/− mouse is sufficient to reduce the formation of atheroscleroses, although the systemic levels of lipoproteins are not corrected (Linton, 1995). The other way around, if APOE +/+ mice are transplanted with −/− bone marrow, these chimeric animals become more prone to develop atheroscleroses, although their serum level of lipoproteins in normal animals because their liver produces sufficient amounts of ApoE. In conclusion, local production of ApoE by tissue macrophages is sufficient to prevent storage of lipoproteins leading to decreased atheroscleroses formation.

### DETAILED DESCRIPTION OF THE INVENTION

The invention provides a gene delivery vehicle comprising an APOE or APOE-like gene construct or functional fragment thereof that is expressed in the host cell to alter or modify the ApoE phenotype of the host cell, more specifically to provide an ApoE phenotype that results in an elevation of the Aβ processing activity of the transfected cell. In a preferred embodiment, a gene delivery vehicle according to the invention is provided with a gene or fragment thereof comprising APOE e2 or APOE e3 or functional fragment thereof. These genes or fragments encode gene products (proteins) that have superior Aβ processing capacity over those of, for example, APOE e4.

In a preferred embodiment, the invention provides a gene delivery vehicle further comprising a secretion signal allowing secretion of a gene product of said gene or fragment thereof. Most of the APOE genes of the invention comprise a secretion signal for transport of the protein to the exterior of the cell, however, additional secretion signals may be added to enhance specific secretion in glial cell progenitors and their progeny.

The invention provides a method for providing a cell with a higher Aβ processing capacity than it had before comprising treating said cell with a gene delivery vehicle according the invention. The cells, preferably glial cell progenitor cells (monocytes or even stem cells) with the altered phenotype, find application in human gene therapy aimed at the reduction of the risk, onset, or development of dementia such as AD.

In this manner, cells such as glial cells are provided that may originate from individuals whose ApoE phenotype enhances the development of AD but are now modified or changed to an ApoE phenotype that decreases the risk on development of AD.

The invention provides a method for providing an individual with a higher Aβ processing capacity than said individual had before comprising providing said individual with at least one cell according to the invention. There are several methods to perform this phenotype switch, two possible examples are:

1) Modification of the committed progenitors of glial cells by a gene delivery vehicle according to the invention such as an adenoviral, AAV, or other viral or non-viral vector as provided by the invention. Monocytes, the progenitors of tissue macrophages/glial cells, are collected from patients by leucophoresis. Upon infection with a gene delivery vehicle according to the invention containing the desired APOE gene or functional fragment thereof, cells are transplanted back into the patient were the cells will migrate into their “end tissues” and differentiate into tissue macrophages/glial cells. Since the cells do not divide, expression of the transgene occurs only during the lifespan of the cell which is 0.5-2 years. This method has to be repeated every year to assure that sufficient numbers of transduced glial cells remain present (>0.5% of all glial cells).

2) Transduction of pluripotent hematopoietic stem cells (HSC) by a gene delivery vehicle according to the invention, such as an integrating retroviral vector containing the desired APOE transgene. HSC are obtained by bone marrow puncture or from the peripheral blood by leucopheresis from “mobilized” patients, for instance, with GM-CSF. Upon purification of the fraction containing the HSC, these cells are transduced, for instance, by co-cultivation with the virus producer cells. Patients are pre-treated, if necessary, with a myeloablative therapy, radiation, chemotherapy or a combination of both (Havenga, 1997). In a preferred embodiment, the invention provides a method of treatment wherein said individual has a known increased risk of development of dementia such as AD, but patients with unknown risks can also be treated, for example, with a cell comprising a gene delivery vehicle comprising an e2 gene or functional fragment thereof.

A preferred retroviral vector is described in International Patent Publication WO 93/07281, containing chimeric LTRs of MoMLV and MoMSV and a mutant polyoma enhancer PyFlOl. Furthermore, the retroviral particle can contain mutant or chimeric envelope proteins that enter the HSC through the human homologue of the murine ecotropic virus receptor. Alternatively, a mutant retroviral envelope is used that is optimized for HSC using envelope display libraries.

With the development of gene therapy it becomes feasible to change the phenotype of mammalian cells by transducing cells with a vector containing a gene of interest. Upon infection, and depending on the vector, the genetic information will be either stably integrated into the host's genome or the vector will remain present as episomal DNA. In both cases, depending on the promoter and/or the locus control region present in the vector, gene transcription can take place.

The invention provides a method to alter the development of dementia by changing the ApoE phenotype of glial cells in the brain from one that gives an increased risk of developing AD to one that gives a normalized or reduced risk of developing AD. This is based on the finding that the presence of APOE-containing monocyte-derived microglial and macroglial cells in the brain is sufficient to delay the formation of congophilic inclusions in APOE knockout mice.

A gene therapy vehicle according to the invention may, for example, comprise an adenovirus-, an adeno-associated virus-, or a retrovirus-derived vector or a non-viral vector for delivery of the APOE gene construct.

The invention is further exemplified in the experimental part of the description, which does not limit the invention.

### EXAMPLES

### Materials and methods

APOE e2 Retroviral Vector

Both ecotropic and amphotropic retroviral vectors containing the APOE e2 cDNA are generated as previously described (Vogels, 1996).

Briefly, the APOE e2 cDNA is cloned into pLEC using a unique restriction site present in the polylinker of pLEC, thus generating IG-APOEe2-1. The sequence of APOE e2 is derived from the sequence given in Genbank (locus HUMAPOE3)by changing the nucleotide C into T at position number 586, as is said in the text given with the sequence. The APOE e2 cDNA is ligated to BamHI-linkers (#1065, Biolabs, Beverly, Mass.), digested with BamHI, and ligated into BamHI-digested pLEC, resulting in the vector IG-APOEe2-1. Subsequently, IG-APOEe2-1 is digested with NheI, which is present in both the 5′- and 3′-LTR of pLEC. The NheI fragment of IG-APOEe2-1 containing the APOE e2 gene is isolated from agarose gel separation technique and purified by using a Gene clean kit (BIO-lOl Inc, Calif., USA). This fragment is cloned into the NheI site of construct pBR.dMo+PyFlOl to generate a retroviral construct coded IG-APOEe2-2. This construct is used to generate recombinant retroviruses in the following manner: Ten mg of pIG-APOEe2-2 and one mg of pCMV-Neo are cotransfected by calcium precipitation (Gibco, according to manufacturers protocol) into GP+E86 ecotropic producer cells. Stably transfected cells are selected by adding 1 mg/ml of G418 and culture the cells for 7 days. G418-resistant cells, as demonstrated by killing of parental GP+E86 cells, are pooled and tested for APOE e2 expression and virus production by detection of ApoE e2 in the producer cells and in NIH/3T3 fibroblasts infected with supernatant generated by these producer cells.

Cell culture supernatant derived from G418-resistant APOE e2-expressing GP+E86 producer cells is used to infect the amphotropic retrovirus producer cell line PA317. This infection is performed by adding 1 ml GP+E86-derived virus supematant to 10⁴ PA317 cells in the presence of 4 mg/ml protamine HCL in a 24-well plate. After 48 hrs the cells are harvested by trypsination. A cloned retroviral producer cell line is obtained by performing two rounds of limiting dilution in 96-well plates. For this purpose, infected PA317 cells are seeded at a concentration of 0.3 cell per well in 100 μl Dulbeco's modified eagles medium (DMEM). Fifty independent clones are screened for expression of ApoE e2 protein in the supernatant of the individual producer clones and in the supernatant of NIH/3T3 cells infected with virus supernatant of this clone. The clone which expresses the highest amount of ApoE e2 and upon infection gives the highest titer of APOE e2 in NIH/3T3s is selected, coded PA317-APOEe2 and used for retrovirus production. Virus supernatant from this clone is also used to infect ecotropic GP+E86 virus producer cells, and the highest expression clone is selected similarly as described above. This clone is coded Gp+E86-APOEe2 and is used to produce an ecotropic recombinant retrovirus batch. A preferred retroviral vector as described in International Patent Publication WO 93/707281 containing chimeric LTR's of MoMLV and MoMSV and a mutant polyoma enhancer PyFlOl. Furthermore the retroviral particle can contain mutant or chimeric envelope proteins that enter the HSC through the human homologue of the murine ecotropic virus receptor. Alternatively, a mutant retroviral envelope is used that is optimized for HSC using envelope display libraries, for example, which enter HSC cells through hCAT1.

Human Umbilical Cord Blood (CB)

CB mononuclear cells (MNC) are separated by a Ficoll density gradient and used for ApoE phenotyping and purification of CD34+ hematopoietic progenitor cells.

CD34+ hematopoietic progenitor cells used for in vitro culture of monocytes are separated by magnetic activated cell sorting (MACS, Miltenyi, Germany) according to the manufacturer's protocol. Primary monocytes are separated by seeding the CD34− cell fraction on tissue culture plastic in DMEM and incubating cells for one hour at 37° C. After one hour the nonadherent cells are removed and the remaining cells are washed twice with phosphate buffered saline (PBS). The remaining cells are mainly monocytes as shown by CD14 positive staining in a Flow cytometric assay.

Transduction of CD34+ cells

10 ⁵ APOE e2-negative CD34+ cells are seeded in a 24 wells plates which contain a monolayer of lethally irradiated 2A317-IG-APOEe2 producer cells in the presence of 10 μg/ml rhulL-3 and rhuGM-CSF and 4 μg/ml protamine HCL.

In this manner cells are differentiated into the myelocytic lineage and at the same time these cells are transduced with the retroviral vector by co-cultivating them with virus producer cells. After 7-14 days the supernatant is harvested to determine APOE e2 expression.

Animals

Homozygous APOE −/− knockout mice we used either from the Jackson Laboratory (Bar Harbor, Me., USA), Hill (Piedrahita, 1992), or Leiden University Medical Center (Leiden, The Netherlands) (van Ree, 1995). Both mice strains are derived from C57BL/6×129 hybrids that are backcrossed in C56BL/6J mice. As APOE +/+ mice and C57BL/6 mice are used, the two strains are, except for the APOE −/− mutation, genetically identical.

Bone Marrow Transplantation

APOE −/− newborn mice, age 1 to 6 days, are lethally irradiated with 5-10 Gy, 3 hours before transplantation with 1.0-10×10⁶ bone marrow cells obtained from APOE +/+ donor mice. Donor mice, age 4-6 weeks, are used as donors to obtain pseudoautologeous (congenic) bone marrow cells.

As control groups newborn APOE −/− mice are transplanted with 1.0-10×10⁶ BM cells from 4-6 weeks old APOE −/− mice and APOE +/+ newborn mice are transplanted with 1.0-10×10⁶ BM cells from 4-6 weeks old APOE +/+ mice. In each group 24 mice are transplanted as described above.

Starting after 6 weeks, and every 4 weeks until week 26, 4 mice of each group were euthanized.

Transduction of Mouse Bone Marrow (BM)

Transduction of murine BM by co-cultivation occurred as described previously (van Beusechem, 1990). Briefly, BM of adult APOE −/− mice is harvested and enriched for progenitors by a metrizamide density gradient (sp.gr.<1.08 g/cm³). One million low density cells are co-cultivated for 72 hrs with a 70% confluent irradiated (20 Gy) monolayer of HCL on ecotropic IG-human ApoE2 or IG-hu NGFR virus producer cells, supplemented with recombinant human IL-1a, recombinant murine IL-3, and 0.4 μg/ml protamine-HCL. After 72 hours cells are collected and injected into sub-lethally irradiated newborn APOE −/− mice. Groups of three mice are euthanized every 4 weeks starting at week 6 until week 24. Brains from these mice are removed and fixated in paraformaldehyde 4% and human APOE e2 in combination with an murine glial cell marker.

Histology

Mice brains are pre-fixated by perfusing the anaesthetised animals with 50 ml PBS followed by 50 ml of 4% paraformaldehyde in 0.05 M phosphate buffer, pH 7.4. The excised brains are cut in 2 mm slices in a coronal mouse brain matrix and reimmersed in the same fixative for 24 hrs. The slices containing sections of the hippocampal area are paraffin embedded in its coronal plane. Eight mm paraffin sections are cut and mounted onto glass slides, and from each slice two slides always are prepared, which are stained with Haematoxylin or Eosin and Periodic Acid-Schiff reagent. Sections for further staining with antibodies are pre-treated with Histomouse blocking kit (Zymed, USA) according to the manufacturer's protocol.

All labeled and stained sections are examined with either an Olympus light microscope or a Zeiss Axiophot with Plan-NeoFLUAR objectives.

Staining of Congophilia

Paraffin embedded coronal sections of 10 mm were stained with Congo red and prepared for fluorescent detection of amyloid deposits as described by Askansas (Askansas, 1993). Slides were viewed in bright field polarized light by epifluorescent illumination, using two filter combinations for fluorescence analyses: 1. fluorescein isothiocyanate (FITC) filters (475- to 495-nm exciter filter, 520-nm barrier filter and 510-nm dichoic filter) or 2. Texas red filters (530- to 585-nm exiter filters, 615-nm barrier filters and 600-nm dichroic filter).

Aβ Staining

Sections are stained as described by Robertson (Robertson, Dutton et al. 1998). Briefly, after microwave retrieval, a wash step with Tris-buffered saline (TBS), and blocking with Histomouse blocking kit, the slides are stained with a specific monoclonal antibody against the 17-24 amino acid sequence of human Aβ. This antibody 4G8 (Senetek, Maryland Heights, Mo., USA) cross-reacts with murine Aβ.

Human and Murine ApoE Staining in Tissue Sections

To show that donor bone marrow-derived glial cells found in the brains of APOE −/− mice are derived from the transplanted hematopoietic progenitors of either the syngeneic transduced or APOE +/+ donor hematopoietic grafts, 5 serial paraffin embedded sections are cut and stained with a mouse monoclonal antibody to human ApoE e2 (F48.1, Research Diagnostics Inc, NJ, USA) or rabbit polyclonal antibody to mouse ApoE (Biodesign International, Kennebunkport, Me.).

Human APOE e2 in Supernatant

APOE e2 expression levels in cell suspension and/or cellular supernatant are determined by Western blotting. For cell-suspensions, all adherent cells derived from each CB sample are collected after washing twice with PBS by scraping and lysing in RIPA (1% NP-40, 0.5% sodium deoxycholate and 0.1% SDS in PBS supplemented with 1 mM phenylmethylsulfonyifluoride and 0.1 mg/ml trypsin inhibitor). After 15 minutes incubation on ice, the lysates are cleared by centrifugation. Protein concentrations are determined by the Bio-Rad protein assay, according to the manufacturers protocol (BioRad). Equal amounts of whole cell extract are fractionated by SDS-PAGE on 10% gels. Proteins are transferred onto Immobilon-P membranes (Millipore) and incubated with the ApoE e2 monoclonal antibody F48.1 (Research Diagnostics Inc, NJ, USA). The secondary labeling is done with a horseradish-peroxidase conjugated goat anti-mouse antibody (BioRad). Both procedures are done according to the protocol provided by Millipore. Antibody complexes are visualized with the ECL detection system according to the manufacturer's protocol (Amersham).

Human ApoE e2 Expression in Permeabilized Cells

Primary monocytes from individual samples are upon removal by scraping fixated and permeabilized as follows: 5×10⁵ cells are washed in ice-cold PBS. Pelleted cells are resuspended in 0.5 ml ice-cold PBS to which 0.5 ml ice-cold 2% phosphate buffered paraformaldehyde (ph 7.2) containing 160 mg/ml L-a-lysophosphatidylcholine is added drop-wise under constant swirling. The cell suspension is kept on ice for 5 min, whereafter the reaction is stopped by adding 2 ml PBS containing 0.5% bovine serum albumin (BSA). Cells are pelleted and resuspended in 100 μl PBS/0.5% BSA, stained with 10 μl (50 μg/ml) antibody to human ApoE e2 (Clone F48.1, Research Diagnostics Inc, NJ, USA) and stored for 30 minutes at +4° C. After washing and resuspending the pellet in 100 μl PBS/0.5% BSA, 10 μl (50 μg/ml) rat anti mouse conjugated with phycoerythrin (PE) (Becton and Dickinson (B+D), CA, USA) is added and the sample stored for 30 minutes at +4° C. After one additional wash step the cell pellet is resuspended in 500 μl PBS/0.5% BSA and analyzed on a FACSort (B+D) according to the manufacturer's protocol. Data is analyzed with the CELLquest acquisition and analyses program (B+D).

Murine Macrophages and Glial Cells

Paraffin embedded coupes of the brain are stained with either a rat monoclonal antibody to mouse macrophage, NOMA-2 (Accurate chemicals) or a polyclonal mouse or rabbit antibody against the astroglial marker GFAP (Zymed, USA) that cross reacts with murine GFAP to detect macrophages and glial cells in the brain.

Secondary Antibody Staining

Sections stained with single and double unlabeled primary antibodies are washed and incubated with the appropriate APAAP or PAP single-stain or APAAP/PAP doublestain visualization kit (DAKO, Glostrup, Denmark). After this procedure all slides are counter stained with hematoxylin.

### Results

CB MNC

FACS analyses of more than 50 individual CB samples revealed that there is a clear difference in expression between ApoE e2 negative monocytes in and ApoE e2 positive monocytes (Table 1).

Upon transduction of CD34+ progenitors-derived monocytes from an APOE e2-donor is found that expression levels are comparable with the wild type level (Table 1).

TABLE 1
ApoE e2 expression in fresh and cultured transduced human
monocytes.

| | untransducted | transduced |
|---|---|---|
| CD14 + monocytes | (peak signal) | (peak signal) |
| fresh | | |
| ApoE e2 − | 5 ± 2 | nd |
| ApoE e2 + | 1000 ± 250 | nd |
| cultured | | |
| APOE E2 − | 7 ± 2 | 1050 ± 260 |
| APOE E2 − (mock infected) | 9 ± 3 | 10 ± 3 |

Effect of Bone Marrow Transplantation on Occurrence of Aβ-Containing Inclusions

Upon examination of the paraffin section of the brains of animals from the three groups by light microscopy, clusters of PAS positive granular structures were regularly found in the brains of the control mice and of the parental strain mice transplanted with APOE −/− BM. Clusters where found predominantly in the hippocampal region. In the APOE −/− mice transplanted with parental strain BM occasional clusters of granules were found but to a much lesser extent than in the control groups (Table 2).

TABLE 2
number of PAS-positive granular structures in the Hippocampal
region.

| | 6 | 10 | 14 | 18 | 22 | 26 |
|---|---|---|---|---|---|---|
| group | weeks | weeks | weeks | weeks | weeks | weeks |
| APOE +/+ | 0.1 | 0.1 | 0.3 | 0.5 | 0.8 | 1.3 |
| → | | | | | | |
| APOE +/+ | | | | | | |
| APOE +/+ | 0.1 | 0.3 | 0.9 | 1.5 | 2.5 | 5 |
| → | | | | | | |
| APOE −/− | | | | | | |
| APOE −/− | 1 | 5 | 10 | 18 | 26 | 43 |
| → | | | | | | |
| APOE −/− | | | | | | |
| APOE −/− | 0.8 | 1.5 | 4 | 8 | 15 | 20 |
| APOE +/+ | | | | | | |

Chimerism and Murine and Human ApoE Expression in the Brain

Analyses of the brainsections from APOE −/− mice transplanted with APOE +/+ BM revealed that ApoE positive monocytes/glial cells are present in the brains of APOE −/− mice transplanted with APOE +/+ BM (Table 3).

These double positive cells weren't seen in APOE −/− mice transplanted with BM from APOE −/− mice. No ApoE negative monocytes/glial cells found in APOE +/+ mice transplanted with APOE +/+ BM.

TABLE 3
percentage of donor-derived glial cells in APOE −/− mice
transplanted with APOE +/+ bone marrow.

| week | % donor derived cells |
|---|---|
| 4 | 4 |
| 8 | 9 |
| 12 | 15 |
| 16 | 22 |
| 20 | 30 |
| 24 | 36 |

Analyses of brainsections of APOE −/− mice transplanted with GP+E86-IG-POe2 transduced APE −/− BM showed similar results as above showing that the human APOEe2 transgene is ecpressed in BM-derived brain glial cells (Table 4).

TABLE 4
percentage of ApoE e2 positive donor cells in APOE −/− mice
transplanted with GP + E86-IG-ApoE2 transduced APOE −/− BM.

| week | % APOE e2 + donor cells |
|---|---|
| 4 | 2 |
| 8 | 5 |
| 12 | 11 |
| 16 | 14 |
| 20 | 20 |
| 24 | 26 |

### REFERENCES

Aleshkov, S. et al. (1997). “Interaction of nascent ApoE2, ApoE3 and ApoE4 isoforms expressed in mammalian cells with amyloid peptide b (1-40). Relevance to Alzheimer's disease.” Biochemistry 36: 10571-10580.

Askansas, V., W. K. Engel, et al. (1993). “Enhanced detection of Congo-red-positive amyloid depositions in -muscle fibers of inclusions body myositis and brain of Alzheimer's disease using fluorescence technique.” Neurology 43: 1265-1267.

Corder, E. H. et al. (1994). “Protective effect of apolipoprotein E type 2 allele for late onset Alzheimer's disease.” Nature Genet 7: 180-184.

de Villiers, W. J. S., van der Westhuyzen, D. R., et al. (1997). “The apolipoprotein E2 (Arg145 Cys) mutation causes autosomal dominant type III hyperlipoproteinemia with incomplete penetrance.”Arterioscler Thromb Vasc Biol 17: 865-872.

Eglitis, M. A. and E. Mezey (1997). “Hematopoietic cells differentiate into both microglia and macroglia in the brains of adult mice.” PNAS 94: 4080-4085.

Havenga, M. (1997). “Retroviral stem cell gene therapy.” Stem Cells 15: 162-179.

Higgins, G. A., C. H. Large, et al. (1997). “Apolipoprotien E and Alzheimer's disease: a review of recent studies.” Pharmocol Biochem Behav 56(4): 675-685.

Hoogerbrugge, P. M. (1988). Bone marrow transplantation for lysosomal enzyme deficiency. Department of Pediatrics. Leiden, University Hospital Leiden.

Linton, M. F., Atkinson, J. B., et al. (1995). “Prevention of atherosclerosis in apolipoprotein E-deficient deficient mice by bone marrow transplantation.” Science 267: 1034-1037.

Pericak-Vance, M. A. et al. (1991). “Linkage studies in familial Alzheimeer's disease: evidence for chromosome 19 linkage.” Am J hum Genet 48: 1034-1050.

Piedrahita, J. A., S. H. Zhang, et al. (1992). “Generation of mice carrying a mutant apolipoprotein E gene inactivated by gene targeting in embryonic stem cells.” PNAS 89: 4471-4475.

Poduri, A., M. Gearing, et al. (.1992.). “Apolipoprotein E4 and beta amyloid in senile plaques and cerebral blood vessels of aged Rhesus monkeys.” Am J Pathol 6: 1183-1187.

Ree, J. H. van den Broek, M. J. Hofker, m. H. Havekse, L. M. (1995). “Atypical xanthomatosis in apolipoprotein E-deficient mice after cholesterol feeding.” Atherosclerosis 112 (2): 237-243.

Robertson, T. A., N. S. Dutton, et al. (1998). “Age-related congophilic inclusions in the brains of apolipoprotein E-deficient mice.” Neuroscience 81(1): 171-180.

Schupf, N., D. Kapell, et al. (1998). “Earlier onset of Alzheimer's disease in men with Down syndrome.” Neurology 50: 991-995.

Strittmater, W. J. et al. (1993). “Apolipoprotein E: high-avidity binding to b-amyloid and increase frequency of type e4 allele in late-onset familial Alzheimer's disease.” PNAS 90: 1977-1981.

Tyrrel, J., M. Cosgrave, et al. (1998). “A protective, effect of apolipoprotein E e2 allele on dementia in Down's syndrome.” Biol Psychiatry 43: 397-400.

van Beusechem, V. W. Kukler, A. Einerhand, M. P. W. Bakx, T. A. van der Eb, A. J. van Bekkum, D. W. and Valerio D. (1990) “Expression of human adenosine deaminase in mice transplanted with hemopoietic stem cells infected with amphotropic retroviruses.” J. Exp. Med. 172: 329-336.

Vogels, R. Boesen, J. van Es, H. H. G van Beuschem, V. W. Valerio, D. (1996). “Improved retroviral vectors especially suitable for gene therapy.” patent application Wo96/35798

Weisgraber, K. H. (1994). “Apolipoprotein E: structure-function relationships.” Adv Prot Chem 45: 249-302.

What is claimed is:

1. A gene delivery vehicle comprising an apolipoprotein E gene or functional fragment thereof having the function of apolipoprotein E gene.

2. The gene delivery vehicle of claim 1 wherein said apolipoprotein E gene comprises:
e2 apolipoprotein E gene, or
e3 apolipoprotein E gene.

3. The gene delivery vehicle of claim 1, said gene delivery vehicle further comprising:
a secretion signal incorporated into said gene delivery vehicle, allowing for secretion of a gene product of said apolipoprotein E gene or said functional fragment thereof.

4. The gene delivery vehicle of claim 2, said gene delivery vehicle further comprising:
a secretion signal incorporated into said gene delivery vehicle, allowing for secretion of a gene product of said apolipoprotein E gene or said functional fragment thereof.

5. The gene delivery vehicle of claim 1, claim 2, claim 3, or claim 4, said gene delivery vehicle adapted to deliver said apolipoprotein E gene or functional fragment thereofto a human cell.

6. The gene delivery vehicle of claim 5 wherein said human cell is a glial-cell progenitor cell.

6. A process for providing a cell with an increased amyloid β processing capacity than the cell had before said process, said method comprising treating the cell with a gene delivery vehicle comprising an apolipoprotein E gene or functional fragment thereof having the function of apolipoprotein E gene.

7. The process according to claim 6 wherein apolipoprotein E gene comprises:
e2 apolipoprotein E gene, or
e3 apolipoprotein E gene.

8. The process according to claim 6, wherein said gene delivery vehicle further comprises:
a secretion signal incorporated into said gene delivery vehicle, allowing for secretion of a gene product of said apolipoprotein E gene or said functional fragment thereof.

9. The process according to claim 7, wherein said gene delivery vehicle further comprises:
a secretion signal incorporated into said gene delivery vehicle, allowing for secretion of a gene product of said apolipoprotein E gene or said functional fragment thereof.

10. The process according to claim 6, claim 7, claim 8, or claim 9, wherein said cell is a human cell.

11. The process according to claim 10 wherein said human cell is a glial-cell progenitor cell.

12. A cell comprising a gene delivery vehicle comprising an apolipoprotein E gene or functional fragment thereof having the function of apolipoprotein E gene.

13. The cell of claim 12 wherein said apolipoprotein E gene comprises:
e2 apolipoprotein E gene, or
e3 apolipoprotein E gene.

14. The cell of claim 12, wherein said gene delivery vehicle further comprises:
a secretion signal incorporated into said gene delivery vehicle, to allow for secretion of a gene product of said apolipoprotein E gene or said functional fragment thereof.

15. The cell of claim 13, wherein said gene delivery vehicle further comprises:
a secretion signal incorporated into said gene delivery vehicle, allowing for secretion of a gene product of said apolipoprotein E gene or said functional fragment thereof.

16. The cell of claim 12 wherein said cell is a human glial-cell progenitor cell or a human glial cell progenitor cell progeny.

17. A method for providing an individual with a higher amyloid β processing capacity than before said individual was provided with the method, said method comprising: providing said individual with at least one cell comprising a gene delivery vehicle comprising an apolipoprotein E gene or functional fragment thereof having the function of apolipoprotein E gene.

18. The method according to claim 17 wherein said apolipoprotein E gene comprises:
e2 apolipoprotein E gene, or
e3 apolipoprotein E gene.

19. The method according to claim 17, wherein said gene delivery vehicle further comprises:
a secretion signal incorporated into said gene delivery vehicle, to allow for secretion of a gene product of said apolipoprotein E gene or said functional fragment thereof.

20. The method according to claim 18, wherein said gene delivery vehicle further comprises:
a secretion signal incorporated into said gene delivery vehicle, allowing for secretion of a gene product of said apolipoprotein E gene or said functional fragment thereof.

21. The method according to claim 18 wherein said cell is a human glial-cell progenitor cell or a human glial cell progenitor cell progeny.

22. The method according to claim 18 wherein said individual is suffering from Alzheimer's disease or has an increased risk of developing dementia.
